(19) United States
(12) Patent Application Publication
(10) Pub. No.: US 2004/0248097 A1
(43) Pub. Date: Dec. 9, 2004

(54) Interleukin-20 variants and promoters

(76) Inventor: Ming-Shi Chang, Tainan (TW)

Correspondence Address: FISH & RICHARDSON PC, 225 FRANKLIN ST, BOSTON, MA, 02110, US

(21) Appl. No.: 10/444,765

(22) Filed: May 23, 2003

(51) Int. Cl. C12Q 1/68; G01N 33/53; G01N 33/567; C07H 21/04

(52) U.S. Cl. 435/6; 435/7.2; 536/23.5

(57) ABSTRACT

Disclosed are human and mouse IL-20 alternatively spliced polypeptides, nucleic acids encoding the polypeptides, and nucleic acids containing a human IL-20 promoter sequence. Also within the scope of this invention are screening, prognostic, therapeutic, and IL-20-activitity-determining methods.

### BACKGROUND

Interleukin-20 (IL-20) is a newly discovered member in the IL-10 family of cytokines. Members in this family are important in controlling inflammation response. Overexpression of IL-20 in transgenic mice leads to neonatal death as well as skin abnormalities, including aberrant epidermal differentiation and the phenotype of psoriasis (Blumberg H et al., Cell 104:9, 2001). The gene expression and biological activity of IL-20 should provide targets for developing new drugs for treating inflammation diseases and skin diseases, as well as new methods for diagnosing these diseases.

### SUMMARY

The present invention is based, at least in part, on the discovery of promoter sequences of the human IL-20 gene, i.e., SEQ ID NOs: 4 and 12. The sequence of SEQ ID NO: 4 is shown immediately below. It corresponds to bp −1957 to −8 of the IL-20 gene, where +1 represents the transcription start of the gene.

| | | | | | | |
|---|---|---|---|---|---|---|
| −1957 | ATGTTTCCAA | GGCGTTGTCT | ATAATTCATC | CCAGGTTCTT | TGAATTTAAT | (SEQ ID NO: 4) |
| −1907 | TTTGTCTGGG | AGCATGGTCC | TACATGATGG | ACTAGTCCCA | GCCTAAAAAA | |
| −1857 | TTTTCCAGTG | TTATTAGGGA | GAAAAGACCA | AGTTCCATGA | AACACAGGAC | |
| −1807 | AACATTAGAT | GCTTTCACTG | CCAAAGAAAA | ATCTGCTATT | GGAAGTCAGA | |
| −1757 | GAAGGGAGGG | ATTGGTATAG | CCCAGAGTTG | TTCAGGAATA | AAGGATTTGG | |
| −1707 | GTTTCATCTT | TAATGATGAC | TAGAGGGAAG | GGTGGCCATG | TTGGAGAGCA | |
| −1657 | ATGGTGGGGA | TAGCAAGGGC | TGAGGAAAAA | CAGCAGGAAT | GAGCCCACAG | |
| −1607 | TGTCTGGAGT | TGTGGAGAAA | CTTGGTTAAG | GGGAACTGGG | TTTAGAGGAG | |
| −1557 | AGAGCAGGAG | AATCCCCCAG | AATTCTCTTC | CAGTTCAGTA | ACTCAGATCT | |
| −1507 | TCTCTATCCA | TCATTTTATC | AAGCAGGAAG | GTGGAGCCAG | AAGGAGGCAC | |
| −1457 | ATCCTTTATC | CACAGCTACA | ACCAGTAATC | CCCTCAGGCA | GTGCTTCTTC | |
| −1407 | CAGGAGGAAG | GTGTTGGGGT | AATCTGTCCT | GCAATTAAGC | TGCTGTAGTG | |
| −1357 | CTTGAGGAAG | AACAATGCCA | CCAGAGAAAT | TCCAAGGGAG | TTCCAGCCCT | |
| −1307 | CACCTGCCTG | AGCTCACTTC | CTTCATGTGA | CATGTATATA | CAGATATAAA | |
| −1257 | TAATGGGAAG | CCTTTCAACT | TGAAACAGGC | TCCTAGGAGA | CCAGAAGCAG | |
| −1207 | CAGCCTTTCC | TGAGCTCAGG | TAAGAGATCT | TACCCTCTAC | TGACACTGCT | |
| −1157 | CACGTTGTTG | TGAGGATCAC | CTACTTCTCC | TAATCATTTA | CCCAGGTATG | |
| −1107 | TTCAAGGTCA | CATCTAAAGG | ACCCTTTTCC | ACGAGGACAA | AATCTCTTTG | |
| −1057 | AGGACAAATA | ATCATCATGT | TTATCTTTGT | ACTTCAGTAC | CTAGCACAAC | |
| −1007 | ATTCAAGACA | GCGGGTGCTC | ATTAAATGCT | CATCAAATTG | TTAGTTCAGG | |
| −957 | ACAACTAACA | TCAATCTCTA | CTTAAAATGA | ATTGATCACT | TGCTCTGTGC | |
| −907 | TAAGTGTATA | AATCATAGAT | TATTGTATTT | AAATAATCGA | TTTAAAATCA | |
| −857 | AAACAATTTC | TGGGTTAAGT | TTAATTATCA | CCATTTTGGG | GTTAAGAAAA | |
| −807 | TTAAACTCAG | AGGTGAGTTG | ACTTGTCCAA | GGTCACATAG | AGGTAGGGTG | |
| −757 | GCCAACTCAT | TCCAGTTTAC | CTGTGGTTTT | TCCAGTTTTA | AAACTGAAAT | |
| −707 | TTTCGTATTT | CAGGAACCAT | TCCCTGCCCC | CCAACCTCAG | TGCTGGGTAA | |
| −657 | ACTGGAATGA | CCCACATCAA | TGGAAACTAG | TAAAGCGAGG | ATTTATTTGG | |
| −607 | ACCCAGTTCT | CTTGTCTCCA | AACCCAGAGT | CCTCTTTGAT | TCTTTTGGGT | |
| −557 | TTGGTTTGCT | TTTTTCCTTT | TCCTACATTT | GACAGTATCT | CGAGTGGTCA | |
| −507 | CAAATGTAAA | AAATGTCTAG | CATATTGCCT | GGCATATAGG | AAAAATTCAG | |
| −457 | TAAGTGATAA | TGATTATCAG | TGCTGTGCCA | AGCTATGGAG | CCAGCCATAT | |
| −407 | ATATATGGAT | GTGTGCATAT | ATATATATGA | TGTGTGTGTA | TATATATATG | |
| −357 | TCTTTATAAA | TTTTATGTAT | TTATTTCTTT | CAAAAATATT | AAAGTATTTG | |
| −307 | AGAAAATTGA | AAAATTAAAA | AGTAGGTTTA | TTACGACTCA | TGACTTTAAG | |
| −257 | TTTAAATATT | TTATTTCTGC | CCCAAACAAA | ATTTATTATA | ATTTTACTGT | |
| −207 | CCTGGTTTTA | AGGGAAGGAA | ACTCATCAAT | AATATTTTCA | TCATATGCTT | |
| −157 | TTGAGAAACA | AAGTTAACCA | TTAAGAATGA | AACATGAAAA | CATGTGAATA | |
| −107 | GTGGTACAAA | TTTTTCCTTT | TGCTTCAATA | TGGCTCAGCA | TGGCACTGTC | |
| −57 | GAATTTTGTC | TTTATATAAA | ATTTTGATAT | TTTGTTTGTC | ATAAGCTTTT | |

Another promoter, SEQ ID NO: 12, has a 318 bp insert (SEQ ID NO: 3; shown below) between bp −169 and −168 of SEQ ID NO: 4 (underlined):

| | | | | | | | |
|---|---|---|---|---|---|---|---|
| TTTTTTTTTT | TTTTTTTTTT | TTTTTTTGAG | ACGGAGTCTC | GCTCTGTCGC | CCAGGCTGGA | GTGCAGTGGC | (SEQ ID NO: 3) |
| GGGATCTCGG | CTCACTGCAA | GCTCCGCCTC | CCGGGTTCAC | GCCATTCTCC | CGCCTCAGCC | TCCCAAGTAG | |
| CTGGGACTAC | AGGCGCCCGC | CACTACGCCC | GGCTAATTTT | TTGTATTTTT | AGTAGAGACG | GGGTTTCACC | |
| GTTTTGGCTG | GGATGGTCTC | GATCTCCTGA | CCTCGTGATC | CGCCCGCCTC | GGCCTCCCAA | AGTGCTGGGA | |
| TTACAGGCGT | GAGCCACCGC | GCCCGGCCTA | ATATTTTC | | | | |

In one aspect, the present invention features a nucleic acid that contains an IL-20 promoter and is operably linked to a reporter gene. The IL-20 promoter contains a fragment of the 318 bp insert-free SEQ ID NO: 4, such as bp −8 to −1456, −8 to −968, −8 to −441, −8 to −286 and −8 to −215 (SEQ ID NOs: 5-9, respectively), or corresponding fragments of the 318 bp insert-containing SEQ ID NO: 12, i.e., SEQ ID NO: 13-17. Alternatively, the IL-20 promoter contains fragment bp −8 to −140 of SEQ ID NO: 4 (SEQ ID NO: 10) or fragment bp −8 to −70 of SEQ ID NO: 4 (SEQ ID NO: 11). Examples of the reporter gene include a LacZ gene, a green fluorescent protein gene, and a luciferase gene.

One can also use a nucleic acid containing an IL-20 promoter operably linked to a reporter gene in a screening method of identifying a compound, e.g., a small molecule compound or a protein, for treating an IL-20-induced disease, such as an inflammation disease or a skin disease. The method includes contacting a compound with a cell containing the just-described nucleic acid; and determining an expression level (i.e., mRNA or protein level) of the reporter gene. The expression level in the presence of the compound, if lower than that in the absence of the compound, indicates that the compound inhibits the activity of the IL-20 promoter and is a candidate for treating an IL-20 induced disease.

In another aspect, the present invention features a prognostic method of determining whether a subject is at risk for developing an IL-20-induced disease. The method includes providing a sample from a subject and determining the presence or absence of the 318-bp insert in the sample. Absence of the 318-bp insert in the sample indicates that the subject is at risk for developing the IL-20 induced disease. The 318-bp insert can be detected by polymerase chain reaction (PCR) amplification or by nucleic acid hybridization.

In yet another aspect, this invention features an isolated nucleic acid containing the sequence of SEQ ID NO: 3 or its complement. It also features an isolated nucleic acid that, under high stringency conditions (i.e., hybridization at 65° C., 0.5× SSC, followed by washing at 45° C., 0.1×SSC), hybridizes to a single stranded probe, the sequence of which contains SEQ ID NO: 3 or its complement. Such a nucleic acid can have at least 40 (e.g., 50, 100, 200, 300, 500, or any number between 40 and 10,000) nucleotides in length. Also within the scope of this invention are an isolated nucleic acid containing one of the sequence SEQ ID NOs: 4-17 or its complement, and an isolated nucleic acid that, under high stringency conditions, hybridizes to a single stranded probe, the sequence of which contains one of SEQ ID NOs: 4-17 or its complement. These nucleic acids can be used in the screening method described above. They can also be used as probes (preferably 40 to 500 bp in length) for detecting the presence or absence of the 318 bp insert in the prognostic method, which is also described above.

An “isolated nucleic acid” is a nucleic acid the structure of which is not identical to that of any naturally occurring nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by PCR amplification, or a restriction fragment; and (d) a recombinant nucleotide sequence, e.g., a nucleotide sequence containing heterologous sequences or encoding a fusion protein.

In still another aspect, this invention features a method of detecting IL-20 activity of a polypeptide. The method includes contacting the polypeptide with a keratinocyte, a monocyte, or a CD8⁺ T cell capable of expressing IL-6 gene or Keratinocyte Growth Factor-1 (KGF-1) gene; and determining an expression level of IL-6 or KGF-1 gene. The expression level in the presence of the polypeptide, if higher than that in the absence of the polypeptide, indicates that the polypeptide has IL-20 activity. Similarly, one can detect IL-20 activity of the polypeptide by contacting it with a monocyte or a CD8⁺ T cell; and determining an expression level of Tumor Necrosis Factor-α (TNF-α) gene in the cell. The expression level of the TNF-α gene in the presence of the polypeptide, if higher than that in the absence of the polypeptide, indicates that the polypeptide has IL-20 activity. One can also use other cells capable of expressing one or more of IL-6, KGF-1, and TNF-α gene, all of which are IL-20 inducible genes, to practice this method. One can also detect IL-20 activity of a polypeptide by contacting the polypeptide with a cell capable of generating reactive oxygen species (ROS) in response to IL-20; and determining a level of the generated ROS. The ROS level in the presence of the polypeptide, if higher than that in the absence of the polypeptide, indicates that the polypeptide has IL-20 activity. These methods allow one to measure the specific activity of an IL-20 preparation enriched or purified from cells expressing IL-20 by recombinant technology.

Also within the scope of this invention are purified polypeptides containing the amino acid sequences of SEQ ID NOs: 1 and 18 shown below (i.e., alternatively spliced variants of human and mouse IL-20). A “purified polypeptide” is a polypeptide free from other biological macromolecules, e.g., it is at least 75% (e.g., 80%, 85%, 90%, 95%, 99%, or any percentage between 70% and 100%) pure by dry weight. Purity can be measured by any appropriate standard method, for example, by column chromatography, polyacrylamide gel electrophoresis, or HPLC analysis. Shown below are the sequences of wild type human (“Wild type”) and mouse IL-20 polypeptides and the two IL-20 spliced variants (“Short form”). The symbol “▾” indicates an intron/exon junction.

| | |
|---|---|
| Human IL-20: | |
| | ▾ |
| Wild type | MKASSLAFSLLSAAFYLLWTPSTGLKTLNLGSCVIATNLQEIRNGFSDIRGSVQAKDGNI |
| Short form | MKASSLAFSLLSAAFYLLWTPSTGLKTLNLGSCVIATNLQEIRNGFSDIRGSVQAKDGNI |
| | ▾ |
| Wild type | DIRILRRTESLQDTKPANRCCLLRHLLRLYLDRVFKNYQTPDHYTLRKISSLANSFLTIK |
| Short form | DIRILRRTESLQDTKPANRCCLLRHLLRLYLDRVFKNYQTPDHYTLRKISSLANSFLTIK |
| | ▾ ▾ |
| Wild type | KDLRLCHAHMTCHCGEEAMKKYSQILSHFEKLEPQAAVVKALGELDILLQWMEETE |
| Short form | KDLRLC ------------------------LEPQAAVVKALGELDILLQWMEETE |
| | (SEQ ID NO:1) |
| Mouse IL-20: | |
| | ▾ |
| Wild type | MKGFGLAFGLFSAVGFLLWTPLTGLKTLHLGSCVITANLQAIQKEFSEIRDSVQAEDTNI |
| Short form | MKGFGLAFGLFSAVGFLLWTPLTGLKTLHLGSCVITANLQAIQKEFSEIRDSV------- |
| | ▾ |
| Wild type | DIRILRTTESLKDIKSLDRCCFLRHLVRFYLDRVFKVYQTPDHHTLRKISSLANSFLIIK |
| Short form | ---------------SLDRCCFLRHLVRFYLDRVFKVYQTPDHHTLRKISSLANSFLIIK |
| | ▾ ▾ |
| Wild type | KDLSVCHSHMACHCGEEAMEKYNQILSHFIELELQAAVVKALGELGILLRWMEEML |
| Short form | KDLSVCHSHMACHCGEEAMEKYNQILSHFIELELQAAVVKALGELGILLRWMEEML |
| | (SEQ ID NO: 18) |

This invention further features isolated nucleic acids containing sequences encoding the above-mentioned two alternatively spliced variants, e.g., SEQ ID NO: 2 or 19. The sequences of SEQ ID NOs: 2 and 19 are shown below:

| | | | | | |
|---|---|---|---|---|---|
| 1 | ctttgaattc | ctagctcctg | tggtctccag | atttcaggcc | taagatgaaa |
| 51 | gcctctagtc | ttgccttcag | ccttctctct | gctgcgtttt | atctcctatg |
| 101 | gactccttcc | actggactga | agacactcaa | tttgggaagc | tgtgtgatcg |
| 151 | ccacaaacct | tcaggaaata | cgaaatggat | tttctgacat | acggggcagt |
| 201 | gtgcaagcca | aagatggaaa | cattgacatc | agaatcttaa | ggaggactga |
| 251 | gtctttgcaa | gacacaaagc | ctgcgaatcg | atgctgcctc | ctgcgccatt |
| 301 | tgctaagact | ctatctggac | agggtattta | aaaactacca | gacccctgac |
| 351 | cattatactc | tccggaagat | cagcagcctc | gccaattcct | ttcttaccat |
| 401 | caagaaggac | ctccggctct | gtctggaacc | tcaggcagca | gttgtgaagg |
| 451 | ctttggggga | actagacatt | cttctgcaat | ggatggagga | gacagaatag |
| 501 | gaggaaagtg | atgctgctgc | taagaatatt | cgaggtcaag | agctccagtc |
| 551 | ttcaatacct | gcagaggagg | catgacccca | aaccaccatc | tctttactgt |
| 601 | actagtcttg | tgctggtcac | agtgtatctt | atttatgcat | tacttgcttc |
| 651 | cttgcatgat | tgtctttatg | catccccaat | cttaattgag | accatacttg |
| 701 | tataagattt | ttgtaatatc | tttctgctat | tggatatatt | tattagttaa |
| 751 | tatatttatt | tattttttgc | tattaatgta | tttaattttt | tacttgggca |
| 801 | tgaaacttta | aaaaaaattc | acaagattat | atttataacc | tgactagagc |

| | |
|---|---|
| 851 | a (SEQ ID NO: 2) |

| | | | | | |
|---|---|---|---|---|---|
| 1 | tgggagacat | cgatagccct | gattgatctc | tttgaatttt | cgcttctggt |
| 51 | ctccaggatc | taggtgtaag | atgaaaggct | ttggtcttgc | ctttggactg |
| 101 | ttctccgctg | tgggttttct | tctctggact | cctttaactg | ggctcaagac |
| 151 | cctccatttg | ggaagctgtg | tgattactgc | aaacctacag | gcaatacaaa |
| 201 | aggaattttc | tgagattcgg | gatagtgtgt | ctttggatag | gtgctgcttc |
| 251 | cttcgtcatc | tagtgagatt | ctatctggac | agggtattca | aagtctacca |
| 301 | gacccctgac | caccataccc | tgagaaagat | cagcagcctc | gccaactcct |
| 351 | ttcttatcat | caagaaggac | ctctcagtct | gtcattctca | catggcatgt |
| 401 | cattgtgggg | aagaagcaat | ggagaaatac | aaccaaattc | tgagtcactt |
| 451 | catagagttg | gaacttcagg | cagcggtggt | aaaggctttg | ggagaactag |
| 501 | gcattcttct | gagatggatg | gaggagatgc | tatagatgaa | agtggagagg |
| 551 | ctgctgagaa | cactcctgtc | caagaatctc | agacctcagc | accatgaaga |
| 601 | catggcccca | ggtgctggca | tttctactca | agagttccag | tcctcagcac |
| 651 | cacgaagatg | gcctcaaacc | accacccctt | tgtgatataa | cttagtgcta |
| 701 | gctatgtgta | tattatttct | acattattgg | ctcccttatg | tgaatgcctt |

| | |
|---|---|
| 751 | catgtgtc (SEQ ID NO: 19) |

In a further aspect, the invention features a method of increasing the expression level of IL-6, TNF-α, or KGF-1, or the level of ROS in a cell by contacting a polypeptide containing the sequence SEQ ID NO: 1 or 18 with the cell. It is known that IL-6, TNF-α, and ROS promote apoptosis. Thus, this method can be used to study apoptosis and pathology of diseases that are associated with deregulation of apoptosis, e.g., cancer. Further, it can also be used to treat a subject suffering from such a disease, i.e., administering to the subject an effective amount of the just-mentioned polypeptide. Since KGF is known to regulate the growth and differentiation of keratinocytes, the method can therefore be used to treat skin diseases and promote wound healing.

The details of one or more embodiments of the invention are set forth in the accompanying description below. Other advantages, features, and objects of the invention will be apparent from the detailed description and the claims.

### DETAILED DESCRIPTION

The present invention relates to IL-20 promoter sequences and alternatively-spliced IL-20 variants. These sequences and variants can be targeted for diagnosing or treating IL-20 related diseases.

For example, within the scope of this invention is a nucleic acid containing a human IL-20 gene promoter sequence, i.e., one of SEQ ID NOs: 3-17. One can identify such a nucleic acid by using any of these sequences as a query to search against a suitable genome database, such as that at National Center for Biotechnology Information (www.ncbi.nlm.nih.gov). Genomic clones containing a nucleic acid thus-identified can be obtained from vendors, e.g., Research Genetics, Inc. (Huntsville, Ala.). The identified nucleic acid can then be isolated by PCR amplification as described in Example 1 below. One can also identify such a nucleic acid by hybridization. More specifically, a labeled nucleic acid probe having all or part of any one of SEQ ID NOs:3-17 can be used to screen an appropriate DNA library (e.g. a human genomic DNA library) for positive clones according to methods well known in the art, e.g., those described in Sambrook et al. Molecular Cloning, 2nd Edition, Cold Spring Harbor Laboratory press, 1989. The nucleic acid can be readily isolated from the positive clones.

The above-described isolated nucleic acid can be used to identify a compound that modulates the expression of IL-20. More specifically, it can be operatively linked to a reporter gene in a vector. One can determine the effect of a test compound on the expression of the reporter gene transfected with this vector. A compound that suppresses the expression is a candidate for treating an IL-20 induced disease. Conversely, a compound that increases the expression is a candidate for treating a disease caused by low levels of IL-20. The test compound can be obtained from compound libraries, such as peptide libraries or peptoid libraries. The libraries can be spatially addressable parallel solid phase or solution phase libraries. See, e.g., Zuckermann et al. J Med Chem 37, 2678-2685, 1994; and Lam Anticancer Drug Des 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J Med Chem 37:2678, 1994; Cho et al. Science 261:1303, 1993; Carrell et al. Angew Chem Int Ed Engi 33:2059, 1994; Carell et al. Angew Chem Int Ed Engl 33:2061, 1994; and Gallop et al. J Med Chem 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J Mol Biol 222:301-310, 1991; and U.S. Pat. No. 5,223,409).

The above-described nucleic acids can be used in a prognostic method. For example, absence of a 318 bp insert (SEQ ID NO: 3) from the IL-20 gene in a subject indicates that the subject is at risk for developing an IL-20 induced disease, including a skin disease (e.g., psoriasis, eczema, atopic dermatitis, and contact dermatitis) and an inflammatory disease (e.g., asthma and bronchitis). To detect the 318 bp in a subject, one can use PCR amplification or hybridization. A PCR amplification method can include the steps of (1) obtaining a cell sample from a subject, (2) isolating DNA from the sample, (3) amplifying the DNA with primers that specifically hybridize to the 318 bp insert or to a region outside the insert, and (4) detecting the presence or absence of an amplification product. The PCR method can also be used for preliminary amplification in conjunction with any of the techniques used for detecting an insert known in the art, e.g., Restriction Fragment Length Polymorphism.

The above-mentioned 318 bp insert can also be detected by a hybridizing method, such as Southern blotting, in situ hybridization. See e.g., Ausubel, F. et al., 3^{rd} eds. Current Protocols in Molecular Biology, John Wiley & Sons New York, 1999. Array-based hybridization methods can also be used. See e.g., Cronin, M. T. et al. Human Mutation 7: 244-255, 996 and Kozal, M. J. et al. (1996) Nature Medicine 2: 753-759, 1996. Arrays suitable for this purpose include a plurality of addresses. Located at these addresses are different probes, such as 318 bp insert-containing sequences (e.g., SEQ ID NO: 12-17) and corresponding 318-bp-free sequences (e.g., SEQ ID NO: 4-9). The latter serve as negative control probes.

As mentioned at the beginning of this section, the present invention also relates to human and mouse IL-20 spliced variant polypeptides (SEQ ID NOs: 1 and 18) and nucleic acids encoding them (SEQ ID NOs: 2 and 19). To prepare a polypeptide containing SEQ ID NO: 1 or 18, a nucleic acid encoding it can be expressed in host cells using recombinant technology. The expressed recombinant polypeptide can be purified from the host cells by methods such as ammonium sulfate precipitation and fractionation column chromatography. See Goeddel, (1990) Gene Expression Technology: Methods in Enzymology 185, Academic Press, San Diego, Calif. The IL-20 activity of the polypeptide can be tested by methods described in Examples 5-7 below.

The above-described IL-20 spliced variant polypeptide can be used to identify a ligand that specifically binds to it. Examples of a ligand of this invention include small molecules (i.e., novel compounds such as novel peptides) and proteins (isolated polyclonal or monoclonal antibodies and receptors). Methods of making antibodies, e.g., monoclonal antibodies, and their fragments are well known in the art. See, for example, Harlow and Lane, Antibodies: A Laboratory Manual, Cold Spring Harbor Laboratory, New York, 1988. The term “antibody” herein refers to intact molecules and their fragments (such as Fab, F(ab′)₂, and Fv), both of which are capable of binding to an epitopic determinant present in the IL-20 spliced variants. One can identify such a ligand as follows. An IL-20 spliced variant polypeptide is immobilized on an affinity matrix for binding with test compounds from a library under suitable conditions. The affinity matrix is then washed with a buffer to remove unbound compounds and non-specifically bound compounds. Compounds that remain bound can be released with an elution buffer. One can further select ligands that specifically bind to the IL-20 spliced variant, but not to a wild type IL-20. These ligands can be further selected for their ability to interfere with the regulation by an IL-20 variant on IL-6, TNF-α, KGF-1, and ROS levels. Such ligands, i.e., antagonists, interfere with the regulation by an IL-20 variant on the downstream IL-20-inducble genes. They therefore can be used to treat diseases induced by IL-20.

One can also treat diseases induced by IL-20 using a compound of this invention that suppresses the activity of an IL-20 promoter. Such a compound can be obtained using the screening method described above. Further, one can treat a dieses associated with low IL-20 levels, and low IL-6, TNF-α, or KGF-1, or ROS levels with a polypeptide containing the sequence SEQ ID NO: 1 or 18.

Thus, also within the scope of this invention are pharmaceutical compositions that contain the above descried active agents and a pharmaceutically acceptable carrier for treating corresponding IL-20 related disorders and a method of using such a composition in an effective amount to treat patents in need thereof. The term “treating” is defined as administration of a composition to a subject, who has an IL-20 related disorder, with the purpose to cure, alleviate, relieve, remedy, prevent, or ameliorate the disorder, the symptom of the disorder, the disease state secondary to the disorder, or the predisposition toward the disorder. An “effective amount” is an amount of the composition that is capable of producing a medically desirable result, e.g., as described above, in a treated subject. The above-described agents can be formulated into dosage forms for different administration routes utilizing conventional methods. For example, such an agent can be formulated in a capsule, a gel seal, or a tablet for oral administration. The pharmaceutical composition can also be administered via the parenteral route. The dosage required depends on the choice of the route of administration; the nature of the formulation; the nature of the subject's illness; the subject's size, weight, surface area, age, and sex; and other drugs being administered. The efficacy of the pharmaceutical composition can be preliminarily evaluated in vitro. For in vivo studies, the composition can be injected into an animal (e.g., the transgenic mouse model described in Blumberg H et al., Cell 104:9, 2001) and its effects on IL-20 induced diseases are then accessed.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Human and mouse IL-20 alternatively spliced variants were obtained and the structures of the human and mouse IL-20 genes were identified.

A panel of human cDNA libraries (kidney, lung, spleen, lymph node, thymus, bone marrow, brain, fetal liver, placenta, heart, testis, liver, and small intestine) was used to isolate human IL-20 cDNA. A full-length human cDNA clone was obtained by repetitive 5′ RACE using anchor primers and gene-specific antisense primers: 5′-GTGGAAGGAGTCCATAGGAG-3′ (first PCR, SEQ ID NO: 22); 5′-AGGAGCTAGGAATTCAAAGAAG-3′ (second PCR, SEQ ID NO: 23). After three rounds of 5′ RACE, the 5′ end of exon 1 containing transcriptional start site was determined. The start site was designated as nucleotide +1. The sequence bp +1 to +1100 (SEQ ID NO: 24) is shown below together with the first amino terminal 14 amino acid residues of human IL-20 encoded by bp 1058-1099 of the sequence.:

| | |
|---|---|
| 1 | TTTTGATATTGCACTAAAATATTTTTATCTTGATGACTGAGGTTTTTTAG |
| 51 | TGCTCCCTTAAATTTTGCACCTAAAATGAGTGCCTCAATTGTTTTACCCT |
| 101 | AACCTCAGCCCATTATTATTTTATCTTAAAACTCAGCAAACACCCTAACC |
| 151 | TGCTCTCTTACTGAGGAGGCTCGCCCAAGAATAAATGAGTTCCGTCATTG |
| 201 | CCTTTCTTCTCTGACTTTTGGGACCATTTGCTTGGTCTAGGACCTGAGTT |
| 251 | GCAGGTCCAGGAAAGCGTGTACTCTCGAATCCACCCAGGAGTGCCTGACT |
| 301 | ACAGTCCTCCTGCAGAGGGCGCTGTGGAGTCCCAGACACGAGTGTTAGGT |
| 351 | GGAATCGGGCTGATTGCCCATCACGTCTTGCCTTTCCCTGGCAGTAGGCT |
| 401 | TGTTATGAAATCATTGACTTTCTATTTGCCTCTGGGGCTTAAGCGAATCT |
| 451 | GTTACCCTCAAATAACCTATCTGATCTCAGACAAATGCCAAACAGAGCTC |
| 501 | AGTTTCTCTGCCCTGTGGGTGGCCATAAAATCCAGACAATTTCCCCCTAG |
| 551 | GTGTTTTCGATGGCGCAGCCACAGCTTCTGTGAGATTCGATTTCTCCCCA |
| 601 | GTTCCCCTGTGGGTCTGAGGGGACCAGAAGGGTGAGCTACGTTGGCTTTC |
| 651 | TGGAAGGGGAGGCTATATGCGTCAATTCCCCAAAACAAGTTTTGACATTT |
| 701 | CCCCTGAAATGTCATTCTCTATCTATTCACTGCAAGTGCCTGCTGTTCCA |
| 751 | GGCCTTACCTGCTGGGCACTAACGGCGGAGCCAGGATGGGGACAGAATAA |
| 801 | AGGAGCCACGACCTGTGCCACCAACTCGCACTCAGACTCTGAACTCAGAC |
| 851 | CTGAAATCTTCTCTTCACGGGAGGCTTGGCAGTTTTTCTTAGTAAGTTGC |
| 901 | GTGGATGGGCCACACTGTCTGAGGCCAGATAAGGCTGTTCTCTTCCCCTG |
| 951 | ACCCCCCACCCCTCACCCCGTGGACACTTGGAGGAGGGGAAACTCAGTAA |
| 1001 | GTCATGCTCTCTTCTTTGAATTCCTAGCTCCTGTGGTCTCCAGATTTCAG |
| 1051 | GCCTAAGATGAAAGCCTCTAGTCTTGCCTTCAGCCTTCTCTCTGCTGCGT |
| | M K A S S L A F S L L S A A |

PCR amplification was used to detect transcripts on the panel of human cDNA libraries with a pair of primers: sense primer (in exon 3): 5′-CTCCAGATTTCAGGCCTAAGATG-3′ (SEQ ID NO: 25), and antisense primer (exon 5): 5′-ATTGAAGACTGGAGCTCTTGACC-3′ (SEQ ID NO: 26).

The results indicated that human IL-20 had two different transcripts. One 607 bp transcript was identical to what was reported previously (accession number AF224266) and named “wild type.” The other transcript variant (532 bp) was new and named “short form” or “alternatively-spliced from.” The wild type was found to be expressed in kidney, lung, and placenta tissues, while the alternatively spliced variant (short form) was found in lung tissue only, indicating that the variant has cell or tissue-type specific functions.

Human IL-20 genomic clone (clone ID, RP1 1-564A8, accession number AF224266) was identified by a homology search against a human high-throughput genome database at National Center for Biotechnology Information (www.ncbi.nlm.nih.gov) using the above-described human IL-20 cDNA sequences as queries. A corresponding BAC clone was purchased from Research Genetics, Inc. (Huntsville, Ala.). A genomic DNA was isolated from it and used in PCR amplification of IL-20 promoter fragments. The genomic sequence was compared with full-length cDNA of IL-20 to locate the exon/intron boundaries. The result indicated that human IL-20 gene contained five exons and four introns and that exon 4 was deleted in the short form of human IL-20.

A pair of mouse IL-20-specific primers was used to amplify mouse IL-20 transcripts on a panel of mouse cDNA libraries. The primers were: sense primer (in exon 1): 5′-GTCTCCAGGATCTAGGTGTAAG-3′ (SEQ ID NO: 27) and antisense primer (in exon 3): 5′-GGCCATGTCTTCATGGTGCTG-3′ (SEQ ID NO: 28).

It was found that mouse IL-20 also had two different tissue-specific transcripts (615 bp and 549 bp). The wild type mouse IL-20 transcript (615 bp) was expressed in brain and heart tissues, while the short variant (549 bp) was expressed in brain tissue only. By comparing the mouse cDNA with genomic sequences, the mouse IL-20 gene was predicted to contain five exons and four introns. Exon 2 was deleted in the mouse IL-20 short form. The amino acid sequences and locations of introns of human and mouse IL-20 spliced variants are shown above in the Summary section.

### EXAMPLE 2

Fragments of human IL-20 promoter were examined for their promoter activities. Eight promoter fragments (A, B, C, D, E, F, G, and H) were amplified by PCR using the above-mentioned human genomic clone (RP11-564A8) as a template. The corresponding 5′ primers and a common antisense primer (bp −8 to −32) and their positions are listed below:

| | | |
|---|---|---|
| primer A: | 5′-ATGTTTCCAAGGCGTTGTCTATA-3′ | (SEQ ID NO: 29; bp −1957 to −1935) |
| primer B: | 5′-TCCTTTATCCACAGCTACAACC-3′ | (SEQ ID NO: 30; bp −1456 to −1435) |
| primer C: | 5′-TAGTTCAGGACAACTAACATCAA-3′ | (SEQ ID NO: 31; bp −966 to −944) |
| primer D: | 5′-TCAGTGCTGTGCCAAGCTATG-3′ | (SEQ ID NO: 32; bp −441 to −421) |
| primer E: | 5′-GTAGGTTTATTACGACTCATGAC-3′ | (SEQ ID NO: 33; bp −286 to −264) |
| primer F: | 5′-TTTACTGTCCTGGTTTTAAGGG-3′ | (SEQ ID NO: 34; bp −215 to −194) |
| primer G: | 5′-CCATTAAGAATGAAACATGAAAAC-3′ | (SEQ ID NO: 35; bp −140 to −117) |
| primer H: | 5′-GCATGGCACTGTCGAATTTTG-3 | (SEQ ID NO: 36; bp −70 to −50) |
| antisense primer: | 5′ AAAAGCTTATGACAAACAAAATATC-3′ | (SEQ ID NO: 37; bp −8 to −32). |

The PCR fragments, all containing at least one TATA box, were subdloned into the Kpn I-Nhe I site of a pGL3 enhancer/promoterless vector (Promega Corp., Madison, Wis.) to generate 8 vectors: pA, pB, pC, pD, pE, pF, pG, and pH. The pGL3 vector contains the entire coding sequences of firefly luciferase gene along with the SV40 enhancer.

Since human kidney expresses IL-20, MDCK cells or human embryonic kidney 293-cells were selected for analyzing promoter activity. The 8 vectors, the promoterless pGL3 plasmid (negative control), and a promoter-containing pGL3 plasmid (positive control) were respectively transfected into MDCK cells. Briefly, cells at a density of 3×10⁵/well in each well of a 6-well plate were transfected with 1 μg of one of the above-mentioned vectors and 0.4 μg of a vector encoding the β-galactosidase (β-gal) gene in the presence of 1 μl of LipofectAMINE 2000 reagent (Invitrogen Corporation: Life Technologies, Inc., Carlsbad, Calif.). The β-gal gene was used as an internal transfection efficiency control. Twenty-four hours after transfection, the medium in each well was replaced with fresh medium. Forty-eight hours after transfection, the cells were collected and the luciferase activity was analyzed according to the protocol of the luciferase assay system (Promega). To obtain relative transfection efficiency, the cell lysate was also analyzed for its β-gal activity. The luciferase activity from each promoter-fusion gene was divided by β-gal activity to normalize against different transfection efficiencies. The results were summarized in Table 1.

TABLE 1
Effects of human IL-20 promoter on luciferase activity

| Vector | Luciferase Activity |
|---|---|
| Positive control | 29,730,303 |
| Negative control | 3,077,426 |
| pA | 30,873,616 |
| pB | 40,722,064 |
| pC | 41,459,428 |
| pD | 46,387,441 |
| pE | 47,013,016 |
| pF | 21,706,121 |
| pG | 18,138,593 |
| pH | 12,300,294 |

As shown in Table 1, all 8 promoter sequences had certain promoter activities. Among them, pE had the highest activity, 14- to 15-fold higher than that of the promoterless pGL3 enhancer vector (the negative control), indicating the presence of enhance sequences in the E region and/or repressor sequences outside the region. This experiment was repeated five times and similar results were obtained.

The luciferase activities were also conducted using human embryonic kidney 293-cells transfected with those vectors listed in Table 1. The activity of each vector in 293 cells was lower than that of MDCK cells, but both types of cells showed luciferase activity with similar trends in these 8 different constructs.

It was found that the 1950 bp IL-20 promoter region (the A region) contained transcription-factor binding sites: several copies of keratinocyte-enhancer, TATA box, NF-κB, AP-1, and C/EBP, which are known to be involved in the transduction pathways of GM-CSF, IL-10, TGF-β, IL-6, and Interferon-γ.

To test if these factors regulate IL-20 promoter activity, MDCK or 293 cells were transfected with pE as described above. 24 hours later, the cells were treated with GM-CSF or IL-10 at the concentration of 20 ng/ml for 24 hours. Luciferase activities of the cells were then measured at the end of incubation as described above. The results were summarized in Table 2.

TABLE 2
Effects of GM-CSF and IL-10 on IL-20 promoter activity

Luciferase Activity

| Vector/factor | 293 cell | MDCK cell |
|---|---|---|
| Positive control (P) | 126,017,622 | 20,947,006 |
| P/GM-CSF | 146,260,497 | 20,054,982 |
| P/IL-10 | 130,442,685 | 16,367,073 |
| Negative control (N) | 5,912,126 | 7,250,574 |
| N/GM-CSF | 6,646,210 | 5,402,745 |
| N/IL-10 | 7,634,640 | 7,435,608 |
| pE | 63,110,801 | 44,912,359 |
| pE/GM-CSF | 108,220,876 | 55,807,102 |
| pE/IL-10 | 103,987,864 | 50,138,164 |

As shown in Table 2, IL-10 and GM-CSF respectively increased IL-20 promoter activity by 72% and 65% in 293 cells, and 24% and 11% in MDCK cells.

To further evaluate whether IL-10 and GM-CSF indeed up-regulated IL-20 transcription, monocytes and 293-cells were treated with either IL-10 or GM-CS. Then the level of IL-20 transcript was analyzed by quantitative real-time PCR. In monocytes, GM-CSF increased IL-20 transcript by 600-fold, while IL-10 increased it by 3-fold; in 293-cells, GM-CSF increased IL-20 transcript by 33% and IL-10 increased it by 3-fold. These results indicated that GM-CSF might function through the production of IL-20.

### EXAMPLE 3

Polymorphism of the promoter of human IL-20 was examined. To do so, a 2102 bp fragment of human IL-20 gene (bp −1957 to +145) was PCR-amplified from each of 140 systemic lupus erythematosus (SLE) patients and 58 healthy controls. It is known that SLE and several other autoimmune diseases, such as rheumatoid arthritis, are associated with polymorphism of the promoter region of IL-10 (a homologue of IL-20) and that high IL-10 production may be a genetic risk factor for disease susceptibility.

Sequence analysis of the 2102 bp fragment revealed four potential single-nucleotide polymorphisms (SNP) at −1728 (S), −666 (S), −32 (R), and +6 (K). Further, PCR products from some individuals contained two fragments: 2420 bp (long) and 2102 bp (short). Sequence analysis of both fragments demonstrated that 2402 bp contained a 318 bp (SEQ ID NO: 3) insert at the position of bp −168.

To characterize effects of the 318 bp insert on human IL-20 gene regulation, five fragments (B′, C′, D′, E′, and F′) were PCR-amplified using the 2420 bp as a template. These 3.18 bp insert-containing fragments were then subeloned into the pGL3 enhancer vector as described above to generate 5 vectors: pB′, pC′, pD′, pE′, and pF′. The promoter activity of each fragment was then analyzed in MDCK cells in the same manner as described above. The results were summarized in Table 3.

TABLE 3
Effect of 318-bp insert on human IL-20 promoter activity

| Vectors | Luciferase Activity |
|---|---|
| Positive control | 29,730,303 |
| Negative control | 3,077,426 |
| pB | 40,722,064 |
| pB′ | 27,747,909 |
| pC | 41,459,428 |
| pC′ | 19,832,662 |
| pD | 46,387,441 |
| pD′ | 12,660,902 |
| pE | 47,013,016 |
| pE′ | 12,708,536 |
| pF | 21,706,121 |
| pF′ | 7,792,873 |

As shown in Table 3, the promoter activities of these 5 fragments were about 32-73% lower than those of B, C, D, E, and F fragments. These results indicated that the 318 bp insert might contain some negative regulatory elements. Indeed, there were 23 copies of SP-1 binding site and one copy of NF-κB binding site in this 318 bp region. SP-1 binding sites in IL-10 gene have been found to play a role in regulating the expression of the gene, the high production of which is associated with autoimmune diseases, and may be a genetic risk factor for disease susceptibility (Gibson, A. W. et al., J. Immunol. 166:3915, 2001). Sequence analysis of the 2102 bp of the human IL-20 promoter also revealed 5 copies of KGF enhancer elements, indicating roles of IL-20 in epidermal differentiation. These elements, as well as the four SNPs, may be associated with IL-20 related diseases.

### EXAMPLE 4

Recombinant IL-20 wild type and spliced variant (short form) proteins were prepared from E. coli cells. cDNAs encoding the human and mouse wild type IL-20 from leucine 25 to leucine 176 were respectively cloned into pET43 vectors (Novagen, Madison, Wis.), which encoded Nus.Tag protein. Similarly, cDNAs encoding the corresponding spliced variants were also cloned in the vectors. The resultant vectors encoded Nus.Tag-IL-20 fusion proteins; the Nus.Tag increased solubility of the proteins in E. coli cells.

The fusion proteins were expressed and purified to >95% by a series of metal chelating chromatography without removing the Nus.Tag part. Mouse IL-20 recombinant fusion proteins were found mostly in the cytosol fraction. Wild type and short form IL-20 fusions showed as two major bands in the regions of 78 and 75 kDa, respectively. The Nus.Tag protein itself was also expressed and purified by chromatography. This protein (65 kDa) was used as a negative control in all biological function analyses as described below.

### EXAMPLE 5

The above-described IL-20 wild type and short form proteins were tested for the ability to stimulate monocytes to produce IL-6 and TNF-α.

Spleen cells were prepared from 8 to 10-week-old male mice and depleted of erythrocytes. The resultant cells were allowed to adhere for 30 min at 37° C., 5% CO₂, and the nonadherent cells were removed by three washes with warm medium. The adherent cells contained >95% monocytes, as determined by Liu's staining, and >98% viable cells.

The monocytes were then cultured at a concentration of 2×10⁶ cells/ml in a 12-well plate, and treated with the above-described mouse recombinant IL-20 wild type or short form proteins (IL-20W and IL-20S, respectively) at a concentration of 100 ng/ml for 8 hours. The fusion partner Nus.Tag (p43) was used as a negative control. LPS, an endotoxin, was used as a positive control. To prove that any production of IL-6 or TNF-α in presence of the recombinant IL-20 proteins was not due to the contamination of LPS, some cells were treated with IL-20 recombinant proteins that had been denatured at 100° C. for 10 min. As LPS cannot be denatured under this condition, a lack of IL-6 or TNF-α production in the presence of the heat-denatured IL-20 proteins (dmIL-20W, dmIL-20S, or dp43) indicated no LPS contamination.

The supernatants of all treated cells were collected and the levels of IL-6 and TNF-α were measured using ELISA kits (R&D, Minneapolis, Minn.) according to the manufacture's directions. Results were summarized in Table 4.

TABLE 4
Effects of mIL-20 on the production of IL-6
and TNF--α by mouse monocytes

| Treatment | IL-6 level pg/ml | TNF- a level pg/ml |
|---|---|---|
| PBS | 63.802 | 133.420 |
| LPS | 337.538 | 431.844 |
| mIL-20W | 163.965 | 199.309 |
| dmIL-20W | 56.971 | 132.800 |
| mIL-20S | 173.722 | 239.017 |
| dmIL-20S | 71.259 | 143.954 |
| p43 | 88.011 | 117.653 |
| dp43 | 60.307 | 134.100 |

As shown in Table 4, monocytes incubated with PBS alone produced low levels of IL-6 and TNF-α. However, the amount of IL-6 and TNF-α increased in the presence of mouse or human IL-20. Both wild type and short form IL-20 showed similar activity. LPS induced monocytes to produce IL-6 and TNF-α, while the heat-denatured proteins did not, suggesting no LPS endotoxin contamination. Furthermore, the fusion partner Nus.Tag p43 alone did not show any activity. These results indicated that the IL-20 wild type and short form proteins had IL-20 activities.

IL-20 proteins purified from mammalian cells were also examined and found to have the same activities as bacterially prepared proteins.

It is known that IL-6 attenuates the synthesis of proinflammatory cytokines while having little effect on the synthesis of anti-inflammatory cytokines, such as IL-10 and transforming growth factor-β. Also, IL-6 promotes the synthesis of IL-Ira and soluble TNF receptor release in humans (Opal, S et al., Chest 117:1162, 2000). Thus, the above results also indicated that, by regulating IL-6 expression, IL-20 plays an important role in inflammation.

### EXAMPLE 6

The IL-20 wild type and short form recombinant proteins were tested for the ability to up-regulate KGF, TNF-α, and IL-6 transcripts in CD8⁺ T cells.

T cells were isolated from the spleens of 8- to 10-week-old BALA/CJ mice. After spleen cells were depleted of erythrocytes and monocytes, T cells were separated from B cells by negative selection using magnetic beads coated with anti-CD19 Ab (Dynal A. S.). CD4⁺ T cells were isolated by incubating the T cells with anti-CD4-coated DETACHaBEAD magnetic bead (Dynal A.S.). CD8⁺ T cells were prepared from the T cells by negative selection of B cells and CD4⁺ T cells.

5×10⁶ CD8⁺ T cells were treated with wild type and short form IL-20 (100 ng/ml), respectively, for 1-8 hours (h). Total RNA was extracted from the cells, and examined for the level of KGF-1 transcript by reverse transcription and real-time PCR. Real-time quantitative PCR was performed using a Lightcycler (Roche) and a LightCycler-Fast Start DNA Master SYBR Green I kit (Roche, Indianapolis, Ind.) according to the manufacturer's instructions. The following specific primers were used:

| | | |
|---|---|---|
| KGF-1 forward: | 5′-ACTCTGCTCTACAGATCAT-3′ | (SEQ ID NO: 38) |
| KGF-1 reverse: | 5′-CCACAATTCCAACTGCC-3′ | (SEQ ID NO: 39) |
| TNF-α forward: | 5′-CCAACGGCATGGATCT-3′ | (SEQ ID NO: 40) |
| TNF-α reverse: | 5′-GGACTCCGCAAAGTCT-3′ | (SEQ ID NO: 41) |
| mIL-6 sense primer: | 5′-TGTGCAATGGCAATTCTGAT-3′ | (SEQ ID NO: 42) |
| mIL-6 antisense primer: | 5′-GGAAATTGGGGTAGGAAGGA-3′ | (SEQ ID NO: 43) |

Mouse β-actin gene was also amplified as an internal control to normalize for RNA amounts. β-actin-specific primes were: sense primer: 5′-GGGAATGGGTCAGAAGGACT-3′ (SEQ ID NO: 44); and antisense primer 5′-TTTGATGTCACGCACGATTT-3′ (SEQ ID NO: 45).

To conduct PCR, cDNA was diluted (1:50) with nuclease-free water, and 2 μl of the resultant solution mixed with the lightcycler SYBR-Green mastermix (Roche): 0.5 μM primers, 3 mM magnesium chloride, and 2 μl Master SYBR-Green, nuclease-free water in a final volume of 20 μl. PCR products were electrophoresed on 1.2% agarose gel or analyzed by a melting-point analysis.

To conduct the melting-point analysis, samples of the PCR products were heated from 50° C. to 95° C., and the decline in fluorescent signal of each sample was assessed. Melting-point characteristics differed between the PCR products. The fluorescence/time-dependent generation of signals was assessed by the manufacturer's software program, and the melting point of each product was matched with its individual melting temperature. Real-time PCR was analyzed using the comparative Ct method according to the manufacturer's instructions. The Ct was inversely proportional to the log amount of template in the PCR. The transcript accumulation index (TAI) was expressed as the fold change between a given sample (Q) and a calibrator (CB), where CB represents a 1-fold expression of each gene. The TAI was calculated as: TAI=_{2ΔΔCt}, where ΔΔCt=(Ct_{Target}-Ct_{β-actin})_{Q}−(Ct_{Untreated}-Ct_{β-actin})_{CB}. The results were summarized in Table 5.

TABLE 5
Effects of mouse IL-20 on KGF-1 and TNF-α transcripts in
CD8+ T cells

| | | TAI (2^{-ΔΔCt}) | | | | |
|---|---|---|---|---|---|---|
| Target transcript | Treatment | 1 h | 2 h | 4 h | 6 h | 8 h |
| KGF-2 | IL-20W | 5.73 | 6.96 | 7.01 | 5.69 | 1.24 |
| | IL-20S | 19.56 | 6.86 | 7.31 | 9.12 | 5.06 |
| | PBS | 1.00 | 1.00 | 1.00 | 1.00 | 1.00 |
| TNF-α | IL-20W | 5.50 | 8.34 | 3.95 | 2.79 | 1.19 |
| | IL-20S | 4.23 | 4.17 | 2.51 | 2.18 | 1.89 |
| | PBS | 1.00 | 1.00 | 1.00 | 1.00 | 1.00 |

As shown in Table 5, both KGF-1 and TNF-α levels increased in CD8⁺ T cells treated with either the wild type or short form IL-20 protein. The level of TNF-α reached the highest level at 2 hours after exposing to the proteins. These results indicated that the IL-20 wild type and short form proteins had IL-20 activities.

The transcript level of IL-6 was also examined in CD8⁺ T cells treated by the mouse IL-20 proteins. The results indicated that both wild type IL-20 and short form IL-20 induced IL-6 level and that wild type IL-20 was more potent than the short form.

### EXAMPLE 7

Wild type IL-20 and short form IL-20 proteins were tested for the ability to induce CD8⁺ T cells to produce reactive oxygen species (ROS). These highly reactive ROS molecules regulate many important cellular events in response to TNF-α, including transcription factor activation (NF-κB), cellular proliferation, and apoptosis. To test this ability, mouse mononuclear cells (B cells, monocytes, and T cells) were used.

B cells were isolated from the spleens of 8- to 10-week-old BALA/CJ mice. After spleen cells were depleted of erythrocytes and monocytes, B cells were separated from T cells by negative selection using magnetic beads coated with anti-CD3 Ab (Dynal A. S., Oslo, Norway). Monocytes and T cells, as well as CD8⁺ T cells, were prepared as described above.

1×10⁶ cells were incubated with mouse IL-20 (100 ng/ml) at 37° C. for 30 min as described above. The cells were then collected and resuspended in 1 ml of RPMI-1640 medium. The ROS activities were determined using the Chemiluminescence Analyzing System (Tohoku Electronic Industrial Co., Sendai, Japan). Chemiluminescence (CL) count was measured according to the manufacturer's directions. After a 100-sec background level determination, 0.5 ml of 25 mM luminol (PBS pH 7.4) was injected into the sample. The CL was monitored continuously for an additional 600 sec. The results were summarized in Table 6.

TABLE 6
Effects of mIL-20 on ROS production in mouse mononuclear cells

| | Total CL count | | | | | | |
|---|---|---|---|---|---|---|---|
| Treatment | LPS | PBS | mIL20-W | mIL20-S | p43 | dmIL20-W | dmIL20-S |
| monocytes | 533,185 | 25,199 | 96,488 | 68,754 | 36,025 | na | na |
| B cells | 300,949 | 141,837 | 112,098 | 111,244 | 99,081 | na | na |
| T cells | 246,516 | 17,769 | 192,411 | 136,743 | 31,153 | 46,695 | 68,602 |
| CD4⁺ T cells | 1,142,649 | 138,246 | 81,111 | 45,566 | 40,948 | na | na |
| CD8⁺ T cells | na | 792,825 | 2,831,221 | 2,193,023 | na | 863,215 | 1,268,193 |

As shown in Table 6, B cells treated with IL-20 for 30 min showed no ROS production, while monocytes showed significant ROS production. An increase in ROS production was also observed in T cells treated with IL-20. Further, only CD8+T cells, but not CD4⁺ T cells, produced more ROS in the presence of IL-20. The results indicated that the mouse IL-20 wild type and shot form proteins induced the ROS levels in monocytes and CD8⁺ T cells.

To analyze the effect of TNF-α antibody on ROS production, the antibody was added to CD8⁺ T cells 30 min before or after the addition of IL-20 (100 ng/ml), or both reagents were added at the same time. After incubating for another 30 min, ROS production from the cells was measured by CL count as described above. The results were summarized in Table 7.

TABLE 7
Effects of TNF-α antibody on ROS production in mouse CD8+ T cells

| | | | mIL- | | | |
|---|---|---|---|---|---|---|
| | | | 20W/TNF-α | mIL-20W + | TNF-α Ab + | |
| Treatment | PBS | mIL-20W | Ab | TNF-α Ab | mIL-20W | TNF-α Ab |
| Total CL count | 792,825 | 2,831,221 | 1,755,277 | 2,568,145 | 1,514,656 | 996,225 |

As shown in Table 7, when CD8⁺ T cells were treated with TNF-α antibody for 30 min followed by IL-20 stimulation for another 30 min (column 6), ROS production was partially inhibited. However, if CD8⁺ T cells were treated with IL-20 for 30 min followed by incubation with TNF-α antibody, ROS production was not inhibited (column 5). If both IL-20 and TNF-α antibody were added at the same time (column 4), the extent of inhibition on ROS production was not as great as when TNF-α antibody was added first. TNF-α antibody alone (column 7) had no effect on ROS production by CD8⁺ T cells. These results indicated that ROS production might not be completely dependent on TNF-α production. CD8⁺ T cells have been found associated with psoriasis, a hyperproliferative and inflammatory skin disorder (Gottlieb S. et al., Nat Med. 1:442, 1995). The results indicated that IL-20 might play roles in the pathogenesis of psoriasis.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A screening method of identifying a compound for treating an IL-20-induced disease, the method comprising:
contacting a compound with a cell containing a nucleic acid comprising an IL-20 promoter operably linked to a reporter gene; and
determining an expression level of the reporter gene in the cell,
wherein the expression level of the reporter gene in the presence of the compound, if lower than that in the absence of the compound, indicates that the compound is a candidate for treating the IL-20 induced disease.

2. The method of claim 1, wherein the IL-20 promoter comprises the sequence SEQ ID NO: 3, 4, 5, 6, 7, 8, 9, 10, 11, 12, 13, 14, 15, 16, or 17.

3. The method of claim 1, wherein the compound is a small molecule compound or a protein.

4. The method of claim 1, wherein the reporter gene is a LacZ gene, a green fluorescent protein gene, or a luciferase gene.

5. The method of claim 1, wherein the IL-20-induced disease is a skin disease or an inflammatory disease.

6. A nucleic acid comprising the sequence SEQ ID NO: 3, 4, 5, 6, 7, 8, 9, 10, 11, 12, 13, 14, 15, 16, or 17, wherein the nucleic acid is operably linked to a reporter gene.

7. The nucleic acid of claim 6, wherein the reporter gene is a LacZ gene, a green fluorescent protein gene, or a luciferase gene.

8. A prognostic method of determining whether a subject is at risk for developing an IL-20-induced disease, the method comprising:
providing a sample from a subject; and
determining the presence or absence of SEQ ID NO: 3 in the sample,
wherein the absence of SEQ ID NO: 3 in the sample indicates that the subject is at risk for developing the IL-20 induced disease.

9. The method of claim 8, wherein the detecting step is conducted by polymerase chain reaction (PCR) amplification.

10. The method of claim 8, wherein the detecting step is conducted by nucleic acid hybridization.

11. An isolated nucleic acid comprising the sequence of SEQ ID NO: 3, or the complement thereof.

12. The nucleic acid of claim 11, wherein the nucleic acid is 318 bp to 10 kb nucleotides in length.

13. An isolated nucleic acid that hybridizes under high stringency conditions to a single stranded probe, the sequence of the probe being SEQ ID NO: 3 or the complement thereof.

14. The nucleic acid of claim 13, wherein the nucleic acid is 40 bp to 10 kb nucleotides in length.

15. An isolated nucleic acid comprising the sequence of SEQ ID NO: 4, 5, 6, 7, 8, 9, 10, 11, 12, 13, 14, 15, 16, or 17, or the complement thereof.

16. The nucleic acid of claim 15, wherein the nucleic acid is 63 bp to 10 kb nucleotides in length.

17. An isolated nucleic acid that hybridizes under high stringency conditions to a single stranded probe, the sequence of the probe being SEQ ID NO: 4, 5, 6, 7, 8, 9, 10, 11, 12, 13, 14, 15, 16, or 17, or the complement thereof.

18. The nucleic acid of claim 17, wherein the nucleic acid is 40 bp to 10 kb nucleotides in length.

19. A purified polypeptide comprising the sequence of SEQ ID NO: 1 or 18.

20. An isolated nucleic acid comprising a sequence encoding the polypeptide of claim 19.

21. The isolated nucleic acid of claim 20, wherein the isolated nucleic acid contains the sequence of SEQ ID NO: 2 or 19.

22. A method of detecting IL-20 activity of a polypeptide, the method comprising:
contacting a polypeptide with a cell capable of expressing IL-6 gene or KGF-1 gene; and
determining an expression level of the IL-6 gene or KGF-1 gene,
wherein the expression level of the IL-6 gene or KGF-1 gene in the presence of the polypeptide, if higher than that in the absence of the polypeptide, indicates that the polypeptide has IL-20 activity.

23. The method of claim 22, wherein the cell is a keratinocyte, a monocyte, or a CD8⁺ T cell.

24. A method of detecting IL-20 activity of a polypeptide, the method comprising:
contacting a polypeptide with a monocyte or a CD8⁺ T cell; and
determining an expression level of the TNF-α gene,
wherein the expression level of the TNF-A gene in the presence of the polypeptide, if higher than that in the absence of the polypeptide, indicates that the polypeptide has IL-20 activity.

25. A method of detecting IL-20 activity of a polypeptide, the method comprising:
contacting a polypeptide with a cell capable of generating more reactive oxygen species in repose to IL-20; and
determining a level of the reactive oxygen species,
wherein the level of the reactive oxygen species in the presence of the polypeptide, if higher than that in the absence of the polypeptide, indicates that the polypeptide has IL-20 activity.

26. A method of increasing the level of IL-6, TNF-α, KGF-1, or reactive oxygen species in a cell, the method comprising contacting a polypeptide of the sequence of SEQ ID NO: 1 or 18 with the cell.


SEQUENCE LISTING (46 sequences)

SEQ ID NO: 1 - protein, 151 aa, Homo sapiens
MKASSLAFSLLSAAFYLLWTPSTGLKTLNLGSCVIATNLQEIRNGFSDIRGSVQAKDGNIDIRILRRTESLQDTKPANRCCLLRHLLRLYLDRVFKNYQTPDHYTLRKISSLANSFLTIKKDLRLCLEPQAAVVKALGELDILLQWMEETE

SEQ ID NO: 2 - DNA, 851 bp, Homo sapiens
ctttgaattcctagctcctgtggtctccagatttcaggcctaagatgaaagcctctagtcttgccttcagccttctctctgctgcgttttatctcctatggactccttccactggactgaagacactcaatttgggaagctgtgtgatcgccacaaaccttcaggaaatacgaaatggattttctgacatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtctggaacctcaggcagcagttgtgaaggctttgggggaactagacattcttctgcaatggatggaggagacagaataggaggaaagtgatgctgctgctaagaatattcgaggtcaagagctccagtcttcaatacctgcagaggaggcatgaccccaaaccaccatctctttactgtactagtcttgtgctggtcacagtgtatcttatttatgcattacttgcttccttgcatgattgtctttatgcatccccaatcttaattgagaccatacttgtataagatttttgtaatatctttctgctattggatatatttattagttaatatatttatttattttttgctattaatgtatttaattttttacttgggcatgaaactttaaaaaaaattcacaagattatatttataacctgactagagca

SEQ ID NO: 3 - DNA, 318 bp, Homo sapiens
tttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttc

SEQ ID NO: 4 - DNA, 1950 bp, Homo sapiens
atgtttccaaggcgttgtctataattcatcccaggttctttgaatttaattttgtctgggagcatggtcctacatgatggactagtcccagcctaaaaaattttccagtgttattagggagaaaagaccaagttccatgaaacacaggacaacattagatgctttcactgccaaagaaaaatctgctattggaagtcagagaagggagggattggtatagcccagagttgttcaggaataaaggatttgggtttcatctttaatgatgactagagggaagggtggccatgttggagagcaatggtggggatagcaagggctgaggaaaaacagcaggaatgagcccacagtgtctggagttgtggagaaacttggttaaggggaactgggtttagaggagagagcaggagaatcccccagaattctcttccagttcagtaactcagatcttctctatccatcattttatcaagcaggaaggtggagccagaaggaggcacatcctttatccacagctacaaccagtaatcccctcaggcagtgcttcttccaggaggaaggtgttggggtaatctgtcctgcaattaagctgctgtagtgcttgaggaagaacaatgccaccagagaaattccaagggagttccagccctcacctgcctgagctcacttccttcatgtgacatgtatatacagatataaataatgggaagcctttcaacttgaaacaggctcctaggagaccagaagcagcagcctttcctgagctcaggtaagagatcttaccctctactgacactgctcacgttgttgtgaggatcacctacttctcctaatcatttacccaggtatgttcaaggtcacatctaaaggacccttttccacgaggacaaaatctctttgaggacaaataatcatcatgtttatctttgtacttcagtacctagcacaacattcaagacagcgggtgctcattaaatgctcatcaaattgttagttcaggacaactaacatcaatctctacttaaaatgaattgatcacttgctctgtgctaagtgtataaatcatagattattgtatttaaataatcgatttaaaatcaaaacaatttctgggttaagtttaattatcaccattttggggttaagaaaattaaactcagaggtgagttgacttgtccaaggtcacatagaggtagggtggccaactcattccagtttacctgtggtttttccagttttaaaactgaaattttcgtatttcaggaaccattccctgccccccaacctcagtgctgggtaaactggaatgacccacatcaatggaaactagtaaagcgaggatttatttggacccagttctcttgtctccaaacccagagtcctctttgattcttttgggtttggtttgcttttttccttttcctacatttgacagtatctcgagtggtcacaaatgtaaaaaatgtctagcatattgcctggcatataggaaaaattcagtaagtgataatgattatcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 5 - DNA, 1449 bp, Homo sapiens
tcctttatccacagctacaaccagtaatcccctcaggcagtgcttcttccaggaggaaggtgttggggtaatctgtcctgcaattaagctgctgtagtgcttgaggaagaacaatgccaccagagaaattccaagggagttccagccctcacctgcctgagctcacttccttcatgtgacatgtatatacagatataaataatgggaagcctttcaacttgaaacaggctcctaggagaccagaagcagcagcctttcctgagctcaggtaagagatcttaccctctactgacactgctcacgttgttgtgaggatcacctacttctcctaatcatttacccaggtatgttcaaggtcacatctaaaggacccttttccacgaggacaaaatctctttgaggacaaataatcatcatgtttatctttgtacttcagtacctagcacaacattcaagacagcgggtgctcattaaatgctcatcaaattgttagttcaggacaactaacatcaatctctacttaaaatgaattgatcacttgctctgtgctaagtgtataaatcatagattattgtatttaaataatcgatttaaaatcaaaacaatttctgggttaagtttaattatcaccattttggggttaagaaaattaaactcagaggtgagttgacttgtccaaggtcacatagaggtagggtggccaactcattccagtttacctgtggtttttccagttttaaaactgaaattttcgtatttcaggaaccattccctgccccccaacctcagtgctgggtaaactggaatgacccacatcaatggaaactagtaaagcgaggatttatttggacccagttctcttgtctccaaacccagagtcctctttgattcttttgggtttggtttgcttttttccttttcctacatttgacagtatctcgagtggtcacaaatgtaaaaaatgtctagcatattgcctggcatataggaaaaattcagtaagtgataatgattatcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 6 - DNA, 961 bp, Homo sapiens
gttagttcaggacaactaacatcaatctctacttaaaatgaattgatcacttgctctgtgctaagtgtataaatcatagattattgtatttaaataatcgatttaaaatcaaaacaatttctgggttaagtttaattatcaccattttggggttaagaaaattaaactcagaggtgagttgacttgtccaaggtcacatagaggtagggtggccaactcattccagtttacctgtggtttttccagttttaaaactgaaattttcgtatttcaggaaccattccctgccccccaacctcagtgctgggtaaactggaatgacccacatcaatggaaactagtaaagcgaggatttatttggacccagttctcttgtctccaaacccagagtcctctttgattcttttgggtttggtttgcttttttccttttcctacatttgacagtatctcgagtggtcacaaatgtaaaaaatgtctagcatattgcctggcatataggaaaaattcagtaagtgataatgattatcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 7 - DNA, 434 bp, Homo sapiens
tcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 8 - DNA, 279 bp, Homo sapiens
gtaggtttattacgactcatgactttaagtttaaatattttatttctgccccaaacaaaatttattataattttactgtcctggttttaagggaaggaaactcatcaataatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 9 - DNA, 208 bp, Homo sapiens
tttactgtcctggttttaagggaaggaaactcatcaataatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 10 - DNA, 133 bp, Homo sapiens
ccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 11 - DNA, 63 bp, Homo sapiens
gcatggcactgtcgaattttgtctttatataaaattttgatattttgtttgtcataagctttt

SEQ ID NO: 12 - DNA, 480 bp, Homo sapiens
ctttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 13 - DNA, 1767 bp, Homo sapiens
tcctttatccacagctacaaccagtaatcccctcaggcagtgcttcttccaggaggaaggtgttggggtaatctgtcctgcaattaagctgctgtagtgcttgaggaagaacaatgccaccagagaaattccaagggagttccagccctcacctgcctgagctcacttccttcatgtgacatgtatatacagatataaataatgggaagcctttcaacttgaaacaggctcctaggagaccagaagcagcagcctttcctgagctcaggtaagagatcttaccctctactgacactgctcacgttgttgtgaggatcacctacttctcctaatcatttacccaggtatgttcaaggtcacatctaaaggacccttttccacgaggacaaaatctctttgaggacaaataatcatcatgtttatctttgtacttcagtacctagcacaacattcaagacagcgggtgctcattaaatgctcatcaaattgttagttcaggacaactaacatcaatctctacttaaaatgaattgatcacttgctctgtgctaagtgtataaatcatagattattgtatttaaataatcgatttaaaatcaaaacaatttctgggttaagtttaattatcaccattttggggttaagaaaattaaactcagaggtgagttgacttgtccaaggtcacatagaggtagggtggccaactcattccagtttacctgtggtttttccagttttaaaactgaaattttcgtatttcaggaaccattccctgccccccaacctcagtgctgggtaaactggaatgacccacatcaatggaaactagtaaagcgaggatttatttggacccagttctcttgtctccaaacccagagtcctctttgattcttttgggtttggtttgcttttttccttttcctacatttgacagtatctcgagtggtcacaaatgtaaaaaatgtctagcatattgcctggcatataggaaaaattcagtaagtgataatgattatcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttctttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 14 - DNA, 1279 bp, Homo sapiens
gttagttcaggacaactaacatcaatctctacttaaaatgaattgatcacttgctctgtgctaagtgtataaatcatagattattgtatttaaataatcgatttaaaatcaaaacaatttctgggttaagtttaattatcaccattttggggttaagaaaattaaactcagaggtgagttgacttgtccaaggtcacatagaggtagggtggccaactcattccagtttacctgtggtttttccagttttaaaactgaaattttcgtatttcaggaaccattccctgccccccaacctcagtgctgggtaaactggaatgacccacatcaatggaaactagtaaagcgaggatttatttggacccagttctcttgtctccaaacccagagtcctctttgattcttttgggtttggtttgcttttttccttttcctacatttgacagtatctcgagtggtcacaaatgtaaaaaatgtctagcatattgcctggcatataggaaaaattcagtaagtgataatgattatcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttctttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 15 - DNA, 752 bp, Homo sapiens
tcagtgctgtgccaagctatggagccagccatatatatatggatgtgtgcatatatatatatgatgtgtgtgtatatatatatgtctttataaattttatgtatttatttctttcaaaaatattaaagtatttgagaaaattgaaaaattaaaaagtaggtttattacgactcatgactttaagtttaaatattttatttctgccccaaacaaaatttattataattttactgtcctggttttaagggaaggaaactcatcaataatattttctttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 16 - DNA, 597 bp, Homo sapiens
gtaggtttattacgactcatgactttaagtttaaatattttatttctgccccaaacaaaatttattataattttactgtcctggttttaagggaaggaaactcatcaataatattttctttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 17 - DNA, 526 bp, Homo sapiens
tttactgtcctggttttaagggaaggaaactcatcaataatattttctttttttttttttttttttttttttttgagacggagtctcgctctgtcgcccaggctggagtgcagtggcgggatctcggctcactgcaagctccgcctcccgggttcacgccattctcccgcctcagcctcccaagtagctgggactacaggcgcccgccactacgcccggctaattttttgtatttttagtagagacggggtttcaccgttttggctgggatggtctcgatctcctgacctcgtgatccgcccgcctcggcctcccaaagtgctgggattacaggcgtgagccaccgcgcccggcctaatattttcatcatatgcttttgagaaacaaagttaaccattaagaatgaaacatgaaaacatgtgaatagtggtacaaatttttccttttgcttcaatatggctcagcatggcactgtcgaattttgtctttatataaaattttgatattttgtttgtcataagctttt

SEQ ID NO: 18 - protein, 154 aa, Mus musculus
MKGFGLAFGLFSAVGFLLWTPLTGLKTLHLGSCVITANLQAIQKEFSEIRDSVSLDRCCFLRHLVRFYLDRVFKVYQTPDHHTLRKISSLANSFLIIKKDLSVCHSHMACHCGEEAMEKYNQILSHFIELELQAAVVKALGELGILLRWMEEML

SEQ ID NO: 19 - DNA, 758 bp, Mus musculus
tgggagacatcgatagccctgattgatctctttgaattttcgcttctggtctccaggatctaggtgtaagatgaaaggctttggtcttgcctttggactgttctccgctgtgggttttcttctctggactcctttaactgggctcaagaccctccatttgggaagctgtgtgattactgcaaacctacaggcaatacaaaaggaattttctgagattcgggatagtgtgtctttggataggtgctgcttccttcgtcatctagtgagattctatctggacagggtattcaaagtctaccagacccctgaccaccataccctgagaaagatcagcagcctcgccaactcctttcttatcatcaagaaggacctctcagtctgtcattctcacatggcatgtcattgtggggaagaagcaatggagaaatacaaccaaattctgagtcacttcatagagttggaacttcaggcagcggtggtaaaggctttgggagaactaggcattcttctgagatggatggaggagatgctatagatgaaagtggagaggctgctgagaacactcctgtccaagaatctcagacctcagcaccatgaagacatggccccaggtgctggcatttctactcaagagttccagtcctcagcaccacgaagatggcctcaaaccaccacccctttgtgatataacttagtgctagctatgtgtatattatttctacattattggctcccttatgtgaatgccttcatgtgtc

SEQ ID NO: 20 - protein, 176 aa, Homo sapiens [sequence omitted]

SEQ ID NO: 21 - protein, 176 aa, Mus musculus
MKGFGLAFGLFSAVGFLLWTPLTGLKTLHLGSCVITANLQAIQKEFSEIRDSVQAEDTNIDIRILRTTESLKDIKSLDRCCFLRHLVRFYLDRVFKVYQTPDHHTLRKISSLANSFLIIKKDLSVCHSHMACHCGEEAMEKYNQILSHFIELELQAAVVKALGELGILLRWMEEML

SEQ ID NO: 22 - DNA, 20 bp, Artificial Sequence
  Primer
gtggaaggagtccataggag

SEQ ID NO: 23 - DNA, 22 bp, Artificial Sequence
  Primer
aggagctaggaattcaaagaag

SEQ ID NO: 24 - DNA, 1100 bp, Homo sapiens
  CDS (1058)...(1099)
ttttgatattgcactaaaatatttttatcttgatgactgaggttttttagtgctcccttaaattttgcacctaaaatgagtgcctcaattgttttaccctaacctcagcccattattattttatcttaaaactcagcaaacaccctaacctgctctcttactgaggaggctcgcccaagaataaatgagttccgtcattgcctttcttctctgacttttgggaccatttgcttggtctaggacctgagttgcaggtccaggaaagcgtgtactctcgaatccacccaggagtgcctgactacagtcctcctgcagagggcgctgtggagtcccagacacgagtgttaggtggaatcgggctgattgcccatcacgtcttgcctttccctggcagtaggcttgttatgaaatcattgactttctatttgcctctggggcttaagcgaatctgttaccctcaaataacctatctgatctcagacaaatgccaaacagagctcagtttctctgccctgtgggtggccataaaatccagacaatttccccctaggtgttttcgatggcgcagccacagcttctgtgagattcgatttctccccagttcccctgtgggtctgaggggaccagaagggtgagctacgttggctttctggaaggggaggctatatgcgtcaattccccaaaacaagttttgacatttcccctgaaatgtcattctctatctattcactgcaagtgcctgctgttccaggccttacctgctgggcactaacggcggagccaggatggggacagaataaaggagccacgacctgtgccaccaactcgcactcagactctgaactcagacctgaaatcttctcttcacgggaggcttggcagtttttcttagtaagttgcgtggatgggccacactgtctgaggccagataaggctgttctcttcccctgaccccccacccctcaccccgtggacacttggaggaggggaaactcagtaagtcatgctctcttctttgaattcctagctcctgtggtctccagatttcaggcctaagatgaaagcctctagtcttgccttcagccttctctctgctgcgt

SEQ ID NO: 25 - DNA, 23 bp, Artificial Sequence
  Primer
ctccagatttcaggcctaagatg

SEQ ID NO: 26 - DNA, 23 bp, Artificial Sequence
  Primer
attgaagactggagctcttgacc

SEQ ID NO: 27 - DNA, 22 bp, Mus musculus
gtctccaggatctaggtgtaag

SEQ ID NO: 28 - DNA, 21 bp, Mus musculus
ggccatgtcttcatggtgctg

SEQ ID NO: 29 - DNA, 23 bp, Homo sapiens
atgtttccaaggcgttgtctata

SEQ ID NO: 30 - DNA, 22 bp, Homo sapiens
tcctttatccacagctacaacc

SEQ ID NO: 31 - DNA, 23 bp, Homo sapiens
tagttcaggacaactaacatcaa

SEQ ID NO: 32 - DNA, 21 bp, Homo sapiens
tcagtgctgtgccaagctatg

SEQ ID NO: 33 - DNA, 23 bp, Homo sapiens
gtaggtttattacgactcatgac

SEQ ID NO: 34 - DNA, 22 bp, Homo sapiens
tttactgtcctggttttaaggg

SEQ ID NO: 35 - DNA, 24 bp, Homo sapiens
ccattaagaatgaaacatgaaaac

SEQ ID NO: 36 - DNA, 21 bp, Homo sapiens
gcatggcactgtcgaattttg

SEQ ID NO: 37 - DNA, 25 bp, Homo sapiens
aaaagcttatgacaaacaaaatatc

SEQ ID NO: 38 - DNA, 19 bp, Artificial Sequence
  Primer
actctgctctacagatcat

SEQ ID NO: 39 - DNA, 17 bp, Artificial Sequence
  Primer
ccacaattccaactgcc

SEQ ID NO: 40 - DNA, 16 bp, Artificial Sequence
  Primer
ccaacggcatggatct

SEQ ID NO: 41 - DNA, 16 bp, Artificial Sequence
  Primer
ggactccgcaaagtct

SEQ ID NO: 42 - DNA, 20 bp, Artificial Sequence
  Primer
tgtgcaatggcaattctgat

SEQ ID NO: 43 - DNA, 20 bp, Artificial Sequence
  Primer
ggaaattggggtaggaagga

SEQ ID NO: 44 - DNA, 20 bp, Artificial Sequence
  Primer
gggaatgggtcagaaggact

SEQ ID NO: 45 - DNA, 20 bp, Artificial Sequence
  Primer
tttgatgtcacgcacgattt

SEQ ID NO: 46 - protein, 14 aa, Homo sapiens
MKASSLAFSLLSAA